(19) United States
(12) Patent Application Publication
(10) Pub. No.: US 2022/0186176 A1
(43) Pub. Date: Jun. 16, 2022

(54) MEDIUM FOR PRIMARY ISOLATION OF PORPHYROMONAS GINGIVALIS, AND A MEDIUM FOR PREPARING THE MEDIUM AND USE THEREOF

(71) Applicant: The First Affiliated Hospital of Henan University of Science and Technology, Luoyang City (CN)

(72) Inventors: Shegan Gao, Luoyang (CN); Bianli Gu, Luoyang (CN); Zijun Lan, Luoyang City (CN); Ke Liu, Luoyang City (CN)

(73) Assignee: The First Affiliated Hospital of Henan University of Science and Technology, Luoyang City (CN)

(21) Appl. No.: 17/508,562

(22) Filed: Oct. 22, 2021

(30) Foreign Application Priority Data
Dec. 14, 2020 (CN) 202011470596.3

(51) Int. Cl. C12N 1/20; C12N 1/02

(52) U.S. Cl. CPC C12N 1/20; C12N 1/02

(57) ABSTRACT

The present disclosure discloses a medium for primary isolation of Porphyromonas gingivalis, and a method for preparing the medium and use thereof. The medium for primary isolation of Porphyromonas gingivalis includes the following components in parts by weight: 10-20 parts of a mixed peptone, 5-10 parts of a yeast extract powder, 1-5 parts of sodium chloride, 10-15 parts of agar, 0.5-1.0 parts of glucose, 0.2-0.8 parts of sodium bicarbonate, 0.1-0.5 parts of an L-cysteine salt, 0.1-0.5 parts of soluble sodium pyrophosphate, 0.0001-0.0005 parts of hemin, 0.00001-0.00005 parts of vitamin K, 500-1000 parts of water and 5-10 parts of a sterile defiberized sheep blood. By adopting the medium for primary isolation of Porphyromonas gingivalis provided by examples of the present disclosure, a culture period of primary isolation of Porphyromonas gingivalis can be greatly shortened, and the primary isolation of Porphyromonas gingivalis can be quickly conducted.

### CROSS REFERENCE TO RELATED APPLICATION(S)

This patent application claims the benefit and priority of Chinese Patent Application No. 202011470596.3 filed on Dec. 14, 2020, the disclosure of which is incorporated by reference herein in its entirety as part of the present application.

### TECHNICAL FIELD

The present disclosure relates to the field of biotechnology, in particular to a medium for primary isolation of a Porphyromonas gingivalis, and a method for preparing the medium and use thereof.

### BACKGROUND ART

Periodontitis is a common chronic inflammatory disease of the oral cavity, and mostly occurs in people over 35 years old. The periodontitis is caused by microbial infections on the gums and periodontal supporting tissues. A “red-complex” is an important factor leading to the periodontitis. Porphyromonas gingivalis (Pg) is a type of gram-negative anaerobic bacteria. Pg is distributed in the oral cavity, digestive system, cardiovascular system, brain and other parts of the human body. According to research reports, as one of the “keystone” bacteria of the “red-complex”, Pg can interact with host cells through various ways to cause inflammation in the lesions. Pg is related to oral inflammation, oral and maxillofacial tumors, gastrointestinal tumors, Alzheimer's disease and other systemic diseases. Pg mainly exists in the oral cavity, and can be detected in periodontal pockets, gingival sulcus, interdental spaces, tongue surface, gums, and other parts. In vitro culture is an important method to study the pathogenicity of Pg, and the periodontitis, gastrointestinal tumors and other related diseases.

In the prior art, the medium for periodontitis-causing anaerobes mostly adopts the tryptic soy broth (TSB) agar, and the Schaedler's agar and the like. These media are rich in nutrients and can culture a relatively-wide spectrum of bacteria; the media can effectively culture not only Pg, but also other non-target competitor bacteria that can greatly hinder the primary isolation of Pg. Moreover, these media also provide abundant nutrients for other non-target bacteria, such that all the requirements of Pg cannot be well met. Furthermore, Pg usually needs to be cultured under anaerobic conditions for 5-7 days to form characteristic black clones. However, the long-term culture also helps the growth of other non-target competitor bacteria, which brings greater difficulties to the primary isolation of Pg. Therefore, the isolation of Pg requires a longer screening cycle.

As a result, it is necessary to provide a medium that can quickly achieve the primary isolation of Porphyromonas gingivalis from many bacteria.

### SUMMARY

A first objective of the present disclosure is to provide a medium for primary isolation of Porphyromonas gingivalis, such that the problems of difficulty and long time for primary isolation of Porphyromonas gingivalis in the prior art can be solved.

A second objective of the present disclosure is to provide a method for preparing the medium for primary isolation of Porphyromonas gingivalis.

A third objective of the present disclosure is to provide a use of the medium for primary isolation of Porphyromonas gingivalis.

The first objective of the present disclosure is implemented by using the following technical solutions: a medium for primary isolation of Porphyromonas gingivalis includes the following components in parts by weight: 10-20 parts of a mixed peptone, 5-10 parts of a yeast extract powder, 1-5 parts of sodium chloride, 10-15 parts of agar, 0.5-1.0 parts of glucose, 0.2-0.8 parts of sodium bicarbonate, 0.1-0.5 parts of an L-cysteine salt, 0.1-0.5 parts of soluble sodium pyrophosphate, 0.0001-0.0005 parts of hemin, 0.00001-0.00005 parts of vitamin K, 500-1000 parts of water and 5-10 parts of a sterile defiberized sheep blood.

In one embodiment, the medium for primary isolation of Porphyromonas gingivalis may include the following components in parts by weight: 15 parts of the mixed peptone, 6 parts of the yeast extract powder, 2.5 parts of the sodium chloride, 15 parts of the agar, 1.0 part of the glucose, 0.4 parts of the sodium bicarbonate, 0.5 parts of the L-cysteine salt, 0.25 parts of the soluble sodium pyrophosphate, 0.0005 parts of the hemin, 0.00005 parts of the vitamin K, 1000 parts of the water and 10 parts of the sterile defiberized sheep blood; or

10 parts of the mixed peptone, 5 parts of the yeast extract powder, 2 parts of the sodium chloride, 10 parts of the agar, 0.5 parts of the glucose, 0.3 parts of the sodium bicarbonate, 0.4 parts of the L-cysteine salt, 0.15 parts of the soluble sodium pyrophosphate, 0.0004 parts of the hemin, 0.00004 parts of the vitamin K, 700 parts of the water and 7 parts of the sterile defiberized sheep blood.

The second objective of the present disclosure is implemented by using the following technical solutions: a method for preparing the medium for primary isolation of Porphyromonas gingivalis includes the following steps:

proportionally mixing the mixed peptone, the yeast extract powder, the sodium chloride, the agar, the glucose, the sodium bicarbonate, the L-cysteine salt, and the soluble sodium pyrophosphate, diluting with the water to 1 L, and mixing well to obtain a mixed solution;

autoclaving the mixed solution and cooling down;

adding a hemin stock solution, a vitamin K stock solution and the sterile defiberized sheep blood proportionally into the cooled mixed solution, and mixing well to obtain a liquid medium; and

pouring the liquid medium onto a plate and cooling to obtain a solid medium.

In one embodiment, the autoclaving the mixed solution may be conducted at 120-125° C. for 10-20 min; and

cooling the mixed solution may specifically include: after autoclaving, placing the mixed solution in the 60° C. water bath when the temperature drops to 65-75° C.

In one embodiment, the method may further include preparing the hemin stock solution before addition, specifically as follows:

uniformly mixing the hemin with K₂HPO₄ in deionized water proportionally, and conducting boiling sterilization to obtain the hemin stock solution.

In one embodiment, based on parts by weight, 0.5 parts of the hemin, 1.74 parts of the K₂HPO₄, and 100 parts of the deionized water may be used.

In one embodiment, the method may further include preparing the vitamin K stock solution before addition, specifically as follows:

uniformly mixing the vitamin K with an absolute ethanol proportionally, and filtering with the 0.45 μm filter membrane to obtain the vitamin K stock solution.

In one embodiment, based on parts by weight, 0.5 parts of the vitamin K, and 100 parts of the absolute ethanol may be used.

In one embodiment, the sterile defiberized sheep blood may be preheated in the 40-45° C. water bath before addition.

The third objective of the present disclosure is implemented by using the following technical solutions: use of the medium for primary isolation of Porphyromonas gingivalis includes:

placing the medium for primary isolation of Porphyromonas gingivalis in a 37° C. incubator, overnight under anaerobic conditions;

sampling, and inoculating samples on the medium for primary isolation of Porphyromonas gingivalis using the divisional continuous streaking method;

placing the inoculated medium for primary isolation of Porphyromonas gingivalis in a 37° C. incubator, and culturing under anaerobic conditions for 7 days;

observing the morphology of colonies on the plate, picking a black circular monoclone into TSB liquid medium for PCR identification; and

for the bacterial solution identified as positive for Porphyromonas gingivalis, continuing inoculating the bacterial solution using the divisional continuous streaking method to another medium for primary isolation of Porphyromonas gingivalis, and culturing under anaerobic conditions until Porphyromonas gingivalis monoclone grows on the plate; where

the anaerobic conditions are: 90% N₂, 5% CO₂, and 5% H₂; and the TSB liquid medium is prepared by adding 30 parts by weight of a TSB and 5 parts by weight of a yeast extract into 1 L of sterile water.

Compared with the prior art, the present disclosure has the following beneficial effects: the mixed peptone may stimulate the growth of bacteria to the greatest extent; the sodium chloride may effectively maintain the osmotic pressure balance of bacteria; the sodium bicarbonate may detoxify the medium; the hemin may effectively promote the production of melanin by anaerobic bacteria; the L-cysteine salt and the soluble sodium pyrophosphate may specifically promote the growth of Porphyromonas gingivalis; the vitamin K and the glucose may provide necessary growth factors for Porphyromonas gingivalis; meanwhile, low-concentration glucose may inhibit the production of high-level acid and alcohol, thereby promoting clone formation. The medium for primary isolation of Porphyromonas gingivalis provided the present disclosure has simple formula and convenient use, may meet the nutritional needs of the growth of Porphyromonas gingivalis, and effectively restrict the growth of other non-target bacteria. Therefore, the culture cycle for primary isolation of Porphyromonas gingivalis is greatly shortened, and high-purity primary Porphyromonas gingivalis for clinical research may be provided in the shortest time.

### BRIEF DESCRIPTION OF THE DRAWINGS

FIG. 1 is a graph showing the results of not inoculating samples on a medium for primary isolation of Porphyromonas gingivalis provided in the Example 1 of the present disclosure.

FIG. 2 is a graph showing the results of inoculating samples on the medium for primary isolation of Porphyromonas gingivalis provided in the Example 1 of the present disclosure.

FIG. 3 is a graph showing the results of inoculating samples on the TSB agar medium added with the hemin, the vitamin K, and the sterile defiberized sheep blood.

FIG. 4 is a graph showing a plate with monoclone of Porphyromonas gingivalis obtained using the medium for primary isolation of Porphyromonas gingivalis provided in the Example 1 of the present disclosure.

FIG. 5 is a graph showing the results of not inoculating samples on a medium for primary isolation of Porphyromonas gingivalis provided in the Example 2 of the present disclosure.

FIG. 6 is a graph showing the results of inoculating samples on the medium for primary isolation of Porphyromonas gingivalis provided in the Example 2 of the present disclosure.

FIG. 7 is a graph showing a plate with monoclone of Porphyromonas gingivalis obtained using the medium for primary isolation of Porphyromonas gingivalis provided in the Example 2 of the present disclosure.

### DETAILED DESCRIPTION OF THE EMBODIMENTS

The present disclosure will be further described below with reference to the accompanying drawings and specific embodiments. It should be noted that, provided that there is no conflict, new embodiments can be formed by arbitrarily combining various embodiments or various technical features described below.

An example of the present disclosure provides a medium for primary isolation of Porphyromonas gingivalis, including the following components in parts by weight: 10-20 parts of a mixed peptone, 5-10 parts of a yeast extract powder, 1-5 parts of sodium chloride, 10-15 parts of agar, 0.5-1.0 parts of glucose, 0.2-0.8 parts of sodium bicarbonate, 0.1-0.5 parts of an L-cysteine salt, 0.1-0.5 parts of soluble sodium pyrophosphate, 0.0001-0.0005 parts of hemin, 0.00001-0.00005 parts of vitamin K, 500-1000 parts of water and 5-10 parts of a sterile defiberized sheep blood. The mixed peptone may stimulate the growth of bacteria to the greatest extent; the sodium chloride may effectively maintain the osmotic pressure balance of bacteria; the sodium bicarbonate may detoxify the medium; the hemin may effectively promote the production of melanin by anaerobic bacteria; the L-cysteine salt and the soluble sodium pyrophosphate may specifically promote the growth of Porphyromonas gingivalis; the vitamin K and the glucose may provide necessary growth factors for Porphyromonas gingivalis; meanwhile, low-concentration glucose may inhibit the production of high-level acid and alcohol, thereby promoting clone formation. The medium for primary isolation of Porphyromonas gingivalis provided by the example of the present disclosure has simple ingredient and convenient use, may meet the nutritional needs of the growth of Porphyromonas gingivalis, and effectively restrict the growth of other non-target bacteria. Porphyromonas gingivalis may quickly form a single typical black characteristic colony, and the problems of Porphyromonas gingivalis being difficult to be distinguished from other melanin-producing anaerobic bacteria, serious pollution of various non-target bacteria and fungi during isolation, and long isolation period may be solved. Therefore, the present disclosure greatly shortens the culture cycle for primary isolation of Porphyromonas gingivalis, and may provide high-purity primary Porphyromonas gingivalis for clinical research in the shortest time.

The present disclosure further provides a method for preparing the medium for primary isolation of Porphyromonas gingivalis, including the following steps:

S1: proportionally mixing the mixed peptone, the yeast extract powder, the sodium chloride, the agar, the glucose, the sodium bicarbonate, the L-cysteine salt, and the soluble sodium pyrophosphate, diluting with the water to 1 L, and mixing well to obtain a mixed solution;

S2: autoclaving the mixed solution and cooling down;

S3: adding a hemin stock solution, a vitamin K stock solution and the sterile defiberized sheep blood proportionally into the cooled mixed solution, and mixing well to obtain a liquid medium; and

S4: pouring the liquid medium onto a plate and cooling to obtain a solid medium.

In one embodiment, the autoclaving the mixed solution in S2 is conducted at 120-125° C. for 10-20 min; and the cooling the mixed solution specifically includes: after autoclaving, placing the mixed solution in a 60° C. water bath when the temperature drops to 65-75° C. After autoclaving, the mixed solution is slowly cooled to 65-75° C., such that the mixed solution may be fully exhausted to avoid forming a large number of bubbles due to rapid cooling.

In one embodiment, S2 further includes S201 specifically as follows: uniformly mixing the hemin with K₂HPO₄ in deionized water proportionally, and conducting boiling sterilization to obtain the hemin stock solution; where S201 is conducted before S3. The hemin stock solution is placed at room temperature for later use. The hemin stock solution is conducive to the stability of the hemin in the preparation of the medium for primary isolation of Porphyromonas gingivalis.

In one embodiment, in S201, based on parts by weight, 0.5 parts of the hemin, 1.74 parts of the K₂HPO₄, and 100 parts of the deionized water are used. One part of the hemin stock solution is added to 1000 parts of the water when adding the hemin stock solution into the mixed solution.

In one embodiment, S2 further includes S202 specifically as follows: uniformly mixing the vitamin K with an absolute ethanol proportionally, and filtering with the 0.45 μm filter membrane to obtain the vitamin K stock solution; where S202 is conducted before S3. The vitamin K stock solution is conducive to the stability of the vitamin K in the preparation of the medium for primary isolation of Porphyromonas gingivalis.

In one embodiment, in S202, based on parts by weight, 0.5 parts of the vitamin K, and 100 parts of the absolute ethanol are used to prepare vitamin K stock solution. 0.1 parts of the vitamin K stock solution is added to 1000 parts of the water when adding the vitamin K stock solution into the mixed solution.

In one embodiment, S2 further includes S203 specifically as follows: preheating the sterile defiberized sheep blood in a 40-45° C. water bath. The sterile defiberized sheep blood is fully preheated before addition to avoid the large temperature difference between the sterile defiberized sheep blood and the medium, which causes the agar to agglomerate. Accordingly, the quality of the medium is improved.

The present disclosure provides a use of the medium for primary isolation of Porphyromonas gingivalis:

placing the medium for primary isolation of Porphyromonas gingivalis in a 37° C. incubator, overnight under anaerobic conditions;

sampling, and inoculating samples on the medium for primary isolation of Porphyromonas gingivalis using a divisional continuous streaking method;

placing the inoculated medium for primary isolation of Porphyromonas gingivalis in a 37° C. incubator, and culturing under anaerobic conditions for 7 days;

observing the morphology of colonies on a plate, picking a black circular monoclone into the tryptic soy broth (TSB) liquid medium for PCR identification; and

for a bacterial solution identified as positive for Porphyromonas gingivalis, continuing inoculating the bacterial solution using the divisional continuous streaking method to another medium for primary isolation of Porphyromonas gingivalis, and culturing under anaerobic conditions until Porphyromonas gingivalis monoclone grows on the plate; wherein

the anaerobic conditions are: 90% N₂, 5% CO₂, and 5% H₂; and the TSB liquid medium is prepared by adding 30 parts by weight of a TSB and 5 parts by weight of a yeast extract into 1 L of sterile water.

By adopting the medium for primary isolation of Porphyromonas gingivalis provided by examples of the present disclosure, a culture period of primary isolation of Porphyromonas gingivalis may be greatly shortened, and the primary isolation of Porphyromonas gingivalis may be quickly conducted.

### Example 1

A medium for primary isolation of Porphyromonas gingivalis included the following components in parts by weight: 15 parts of a mixed peptone, 6 parts of a yeast extract powder, 2.5 parts of sodium chloride, 15 parts of agar, 1.0 part of glucose, 0.4 parts of sodium bicarbonate, 0.5 parts of an L-cysteine salt, 0.25 parts of soluble sodium pyrophosphate, 0.0005 parts of hemin, 0.00005 parts of vitamin K, 1000 parts of water and 10 parts of a sterile defiberized sheep blood.

A method for preparing the medium for primary isolation of Porphyromonas gingivalis included the following steps:

S1: the mixed peptone, the yeast extract powder, the sodium chloride, the agar, the glucose, the sodium bicarbonate, the L-cysteine salt, and the soluble sodium pyrophosphate were proportionally mixed, diluted with the water to 1 L, and mixed well to obtain a mixed solution;

S2: the mixed solution was autoclaved in a autoclave at 121° C. for 15 min, and transferred to the 60° C. water bath to slowly cool down after the autoclave finished the autoclaving and the temperature dropped to 70° C.;

S201: 0.5 parts of the hemin was uniformly mixed with 1.74 parts of K₂HPO₄ in 100 parts of deionized water, sterilization was conducted at 121° C. for 15 min to obtain a hemin stock solution, and the hemin stock solution was placed at room temperature to cool down for later use;

S202: 0.5 parts of the vitamin K was uniformly mixed with 100 parts of an absolute ethanol, filtered with the 0.45 μm filter membrane to obtain a vitamin K stock solution, and the vitamin K stock solution was stored away from light for later use;

S203: the sterile defiberized sheep blood stored at 4° C. was put into the 42° C. water bath for preheating;

S3: the hemin stock solution obtained in S201 according to an amount of 1 part/1000 parts of water and the vitamin K stock solution obtained in S202 according to an amount of 0.1 parts/1000 parts of water were added to the cooled mixed solution obtained in S2; meanwhile, the preheated sterile defiberized sheep blood obtained in S203 was proportionally added to the cooled mixed solution obtained in S2, and the mixed solution was shaken gently to mix well to obtain a liquid medium;

S4: the liquid medium was poured into a plate to prepare a blood agar plate with a thickness of about 10 cm, and placed at room temperature until the liquid medium was sufficiently cooled to obtain a solid medium, namely the medium for primary isolation of Porphyromonas gingivalis.

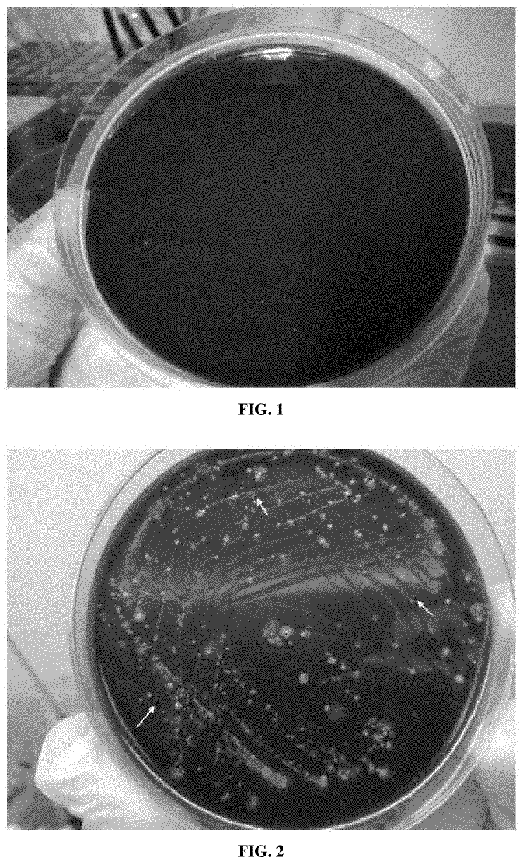

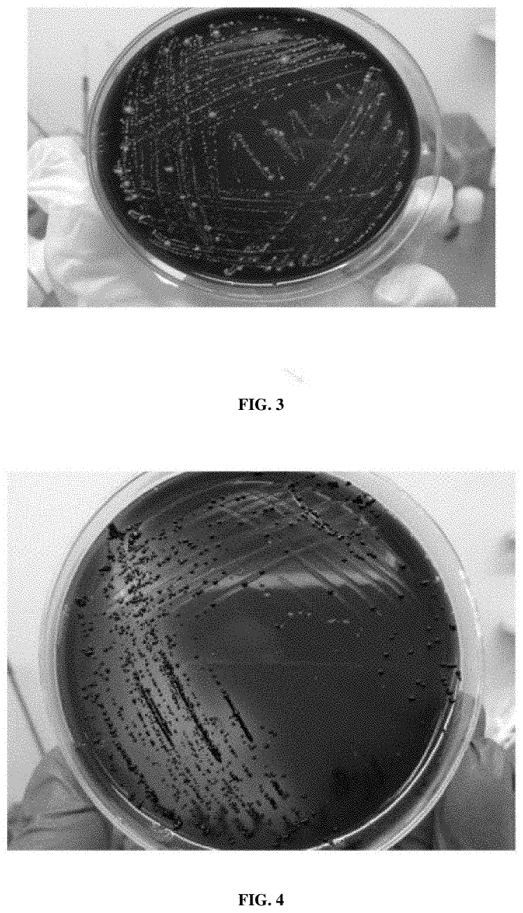

The obtained medium for primary isolation of Porphyromonas gingivalis was placed in a 37° C. incubator, overnight under anaerobic conditions (90% N₂, 5% CO₂, and 5% H₂); a sample was taken from the subject's oral cavity and placed in a TSB liquid medium (the TSB liquid medium was prepared by adding 30 parts by weight of a TSB and 5 parts by weight of a yeast extract to 1 L of sterile water), and the TSB liquid medium was shaken to mix well and instantaneously centrifuged. An appropriate amount of the sample was dipped with an inoculating loop, and inoculated into the medium for primary isolation of Porphyromonas gingivalis provided by the example of the present disclosure and a TSB agar medium added with the hemin, the vitamin K, and the sterile defiberized sheep blood using divisional continuous streaking method. A medium for primary isolation of Porphyromonas gingivalis without inoculation of the sample was regarded as a control. The above three media were incubated in an anaerobic incubator (90% N₂, 5% CO₂, and 5% H₂) at 37° C. for 7 days. Bacterial clones on the plate were observed. FIG. 1 is a graph showing the results of not inoculating the samples, FIG. 2 is a graph showing the results of inoculating the samples on the medium for primary isolation of Porphyromonas gingivalis provided in the example of the present disclosure, and FIG. 3 is a graph showing the results of inoculating the samples to the TSB agar medium added with the hemin, the vitamin K, and the sterile defiberized sheep blood. It can be seen from the experimental results that, the medium for primary isolation of Porphyromonas gingivalis provided by the example of the present disclosure has grown black characteristic clones, and the medium not inoculating the samples and the TSB agar medium added with the hemin, the vitamin K, and the sterile defiberized sheep blood fails to screen out black characteristic clones.

The black clone was identified by bacterial solution polymerase chain reaction (PCR), and Pg 16S rRNA coding sequence was specifically amplified. After being confirmed to be positive, the bacterial solution was inoculated to the medium for primary isolation of Porphyromonas gingivalis provided by the example of the present disclosure by divisional continuous streaking method, and the medium for primary isolation of Porphyromonas gingivalis provided by the example of the present disclosure without inoculation of the bacterial solution was regarded as a control. The bacterial solution was cultured for 7 days in an anaerobic incubator (90% N₂, 5% CO₂, and 5% H₂) at 37° C. The bacterial clones on the plate were observed. FIG. 4 is a graph showing a plate with monoclone of Porphyromonas gingivalis obtained using the medium for primary isolation of Porphyromonas gingivalis provided in the example of the present disclosure. It can be seen that a single jet-black circular characteristic clone is formed on the plate.

It can be seen from the example that, the medium for primary isolation of Porphyromonas gingivalis provided the present disclosure can meet the nutritional needs of the growth of Porphyromonas gingivalis, and greatly shortens the culture cycle for primary isolation of Porphyromonas gingivalis.

### Example 2

The present example differs from Example 1 in that the amount of each component contained in the medium for primary isolation of Porphyromonas gingivalis is different.

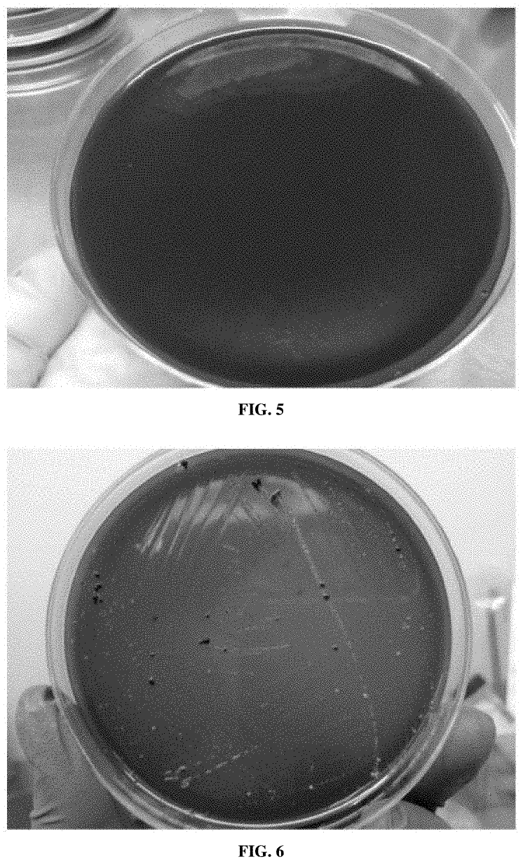

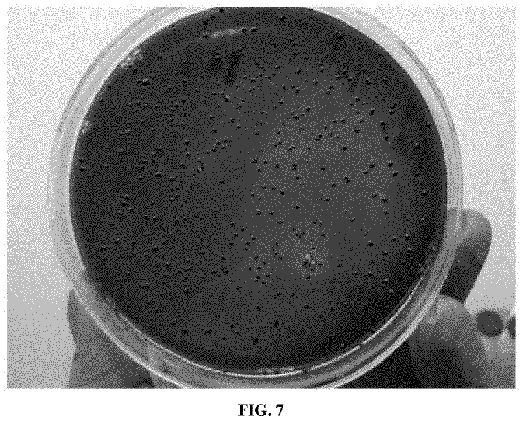

Specifically, the medium for primary isolation of Porphyromonas gingivalis included the following components in parts by weight: 10 parts of a mixed peptone, 5 parts of a yeast extract powder, 2.0 parts of sodium chloride, 10 parts of agar, 0.5 parts of glucose, 0.3 parts of sodium bicarbonate, 0.4 parts of an L-cysteine salt, 0.15 parts of soluble sodium pyrophosphate, 0.0004 parts of hemin, 0.00004 parts of vitamin K, 700 parts of water and 7 parts of a sterile defiberized sheep blood. A method for preparing the medium for primary isolation of Porphyromonas gingivalis and a method for screening Porphyromonas gingivalis by primary isolation were the same as those in the Example 1, which was not described redundantly here. FIG. 5 is a graph showing the results of not inoculating samples, and black characteristic clones are not screened out. FIG. 6 is a graph showing the results of inoculating samples on the medium for primary isolation of a Porphyromonas gingivalis provided in the example of the present disclosure, and black characteristic clones are grown. FIG. 7 is a graph showing a plate with monoclone of Porphyromonas gingivalis obtained using the medium for primary isolation of Porphyromonas gingivalis provided in the example of the present disclosure, and single jet-black circular characteristic clones are formed on the plate.

It can be seen from the example that, the medium for primary isolation of Porphyromonas gingivalis provided the present disclosure can meet the nutritional needs of the growth of Porphyromonas gingivalis, and greatly shortens the culture cycle for primary isolation of Porphyromonas gingivalis.

While the above-described embodiments are merely preferred embodiments of the present disclosure, it is not intended to limit the scope of the present disclosure, and any insubstantial changes and replacements made by those skilled in the art on the basis of the present disclosure fall within the protection scope of the present disclosure.

1. A medium for primary isolation of Porphyromonas gingivalis, comprising the following components in parts by weight:
10-20 parts of a mixed peptone, 5-10 parts of a yeast extract powder, 1-5 parts of sodium chloride, 10-15 parts of agar, 0.5-1.0 parts of glucose, 0.2-0.8 parts of sodium bicarbonate, 0.1-0.5 parts of an L-cysteine salt, 0.1-0.5 parts of soluble sodium pyrophosphate, 0.0001-0.0005 parts of hemin, 0.00001-0.00005 parts of vitamin K, 500-1000 parts of water and 5-10 parts of sterile defiberized sheep blood.

2. The medium for primary isolation of Porphyromonas gingivalis according to claim 1, comprising the following components in parts by weight:
15 parts of the mixed peptone, 6 parts of the yeast extract powder, 2.5 parts of the sodium chloride, 15 parts of the agar, 1.0 part of the glucose, 0.4 parts of the sodium bicarbonate, 0.5 parts of the L-cysteine salt, 0.25 parts of the soluble sodium pyrophosphate, 0.0005 parts of the hemin, 0.00005 parts of the vitamin K, 1000 parts of the water and 10 parts of the sterile defiberized sheep blood; or
10 parts of the mixed peptone, 5 parts of the yeast extract powder, 2 parts of the sodium chloride, 10 parts of the agar, 0.5 parts of the glucose, 0.3 parts of the sodium bicarbonate, 0.4 parts of the L-cysteine salt, 0.15 parts of the soluble sodium pyrophosphate, 0.0004 parts of the hemin, 0.00004 parts of the vitamin K, 700 parts of the water and 7 parts of the sterile defiberized sheep blood.

3. A method for preparing a medium for primary isolation of Porphyromonas gingivalis comprising the following steps:
proportionally mixing a mixed peptone, yeast extract powder, sodium chloride, agar, glucose, sodium bicarbonate, L-cysteine salt, and soluble sodium pyrophosphate, diluting with water to 1 L, and mixing well to obtain a mixed solution;
autoclaving the mixed solution and cooling down, to obtain a cooled mixed solution;
adding a hemin stock solution, a vitamin K stock solution and the sterile defiberized sheep blood proportionally into the cooled mixed solution, and mixing well to obtain a liquid medium; and
pouring the liquid medium onto a plate and cooling to obtain a solid medium comprising 10-20 parts of the mixed peptone, 5-10 parts of the yeast extract powder, 1-5 parts of the sodium chloride, 10-15 parts of the agar, 0.5-1.0 parts of the glucose, 0.2-0.8 parts of the sodium bicarbonate, 0.1-0.5 parts of the L-cysteine salt, 0.1-0.5 parts of the soluble sodium pyrophosphate, 0.0001-0.0005 parts of the hemin, 0.00001-0.00005 parts of the vitamin K, 500-1000 parts of water and 5-10 parts of the sterile defiberized sheep blood.

4. The method according to claim 3, wherein the autoclaving the mixed solution is conducted at 120-125° C. for 10-20 min; and
the cooling the mixed solution specifically comprises: after autoclaving, placing the mixed solution in the 60° C. water bath when the temperature drops to 65-75° C.

5. The method according to claim 3, further comprising preparing the hemin stock solution before addition, specifically as follows:
uniformly mixing the hemin with K₂HPO₄ in deionized water proportionally, and conducting boiling sterilization to obtain the hemin stock solution.

6. The method according to claim 5, wherein based on parts by weight, 0.5 parts of the-hemin, 1.74 parts of the K₂HPO₄, and 100 parts of the deionized water are used.

7. The method according to claim 3, further comprising preparing the vitamin K stock solution before addition, specifically as follows:
uniformly mixing the vitamin K with an absolute ethanol proportionally, and filtering with the 0.45 μm filter membrane to obtain the vitamin K stock solution.

8. The method according to claim 7, wherein based on parts by weight, 0.5 parts of the vitamin K, and 100 parts of the absolute ethanol are used.

9. The method according to claim 3, wherein the sterile defiberized sheep blood is preheated in a 40-45° C. water bath kettle before addition.

10. A method for culturing Porphyromonas gingivalis, comprising:
placing a medium for primary isolation of Porphyromonas gingivalis in a 37° C. incubator, overnight under anaerobic conditions, the medium for primary isolation of Porphyromonas gingivalis comprising 10-20 parts of a mixed peptone, 5-10 parts of a yeast extract powder, 1-5 parts of sodium chloride, 10-15 parts of agar, 0.5-1.0 parts of glucose, 0.2-0.8 parts of sodium bicarbonate, 0.1-0.5 parts of an L-cysteine salt, 0.1-0.5 parts of soluble sodium pyrophosphate, 0.0001-0.0005 parts of hemin, 0.00001-0.00005 parts of vitamin K, 500-1000 parts of water and 5-10 parts of sterile defiberized sheep blood;
sampling, and inoculating samples on the medium for primary isolation of Porphyromonas gingivalis;
placing the inoculated medium for primary isolation of Porphyromonas gingivalis in a 37° C. incubator, and culturing under anaerobic conditions for 7 days;
observing the morphology of colonies on a plate, picking a black circular monoclone into a tryptic soy broth (TSB) liquid medium for PCR identification; and
for a bacterial solution identified as positive for Porphyromonas gingivalis, continuing inoculating the bacterial solution using the divisional continuous streaking method to another medium for primary isolation of Porphyromonas gingivalis, and culturing under anaerobic conditions until Porphyromonas gingivalis monoclone grows on the plate; wherein
the anaerobic conditions are: 90% N₂, 5% CO₂, and 5% H₂; and the TSB liquid medium is prepared by adding 30 parts by weight of a TSB and 5 parts by weight of a yeast extract into 1 L of sterile water.

11. The method according to claim 3, wherein the solid medium comprises the following components in parts by weight:
15 parts of the mixed peptone, 6 parts of the yeast extract powder, 2.5 parts of the sodium chloride, 15 parts of the agar, 1.0 part of the glucose, 0.4 parts of the sodium bicarbonate, 0.5 parts of the L-cysteine salt, 0.25 parts of the soluble sodium pyrophosphate, 0.0005 parts of the hemin, 0.00005 parts of the vitamin K, 1000 parts of the water and 10 parts of the sterile defiberized sheep blood; or
10 parts of the mixed peptone, 5 parts of the yeast extract powder, 2 parts of the sodium chloride, 10 parts of the agar, 0.5 parts of the glucose, 0.3 parts of the sodium bicarbonate, 0.4 parts of the L-cysteine salt, 0.15 parts of the soluble sodium pyrophosphate, 0.0004 parts of the hemin, 0.00004 parts of the vitamin K, 700 parts of the water and 7 parts of the sterile defiberized sheep blood.

12. The method according to claim 10, wherein the medium comprises the following components in parts by weight:
15 parts of the mixed peptone, 6 parts of the yeast extract powder, 2.5 parts of the sodium chloride, 15 parts of the agar, 1.0 part of the glucose, 0.4 parts of the sodium bicarbonate, 0.5 parts of the L-cysteine salt, 0.25 parts of the soluble sodium pyrophosphate, 0.0005 parts of the hemin, 0.00005 parts of the vitamin K, 1000 parts of the water and 10 parts of the sterile defiberized sheep blood; or
10 parts of the mixed peptone, 5 parts of the yeast extract powder, 2 parts of the sodium chloride, 10 parts of the agar, 0.5 parts of the glucose, 0.3 parts of the sodium bicarbonate, 0.4 parts of the L-cysteine salt, 0.15 parts of the soluble sodium pyrophosphate, 0.0004 parts of the hemin, 0.00004 parts of the vitamin K, 700 parts of the water and 7 parts of the sterile defiberized sheep blood.
